(19) United States
(12) Patent Application Publication
(10) Pub. No.: US 2006/0196499 A1
(43) Pub. Date: Sep. 7, 2006

(54) Scuba diver surface location, navigational and communication device and method

(76) Inventor: Kenneth Peter Cannizzaro, Los Gatos, CA (US)

Correspondence Address: STEVEN A. NIELSEN;ALLMAN & NIELSEN, P.C, 100 Larkspur Landing Circle, Suite 212, LARKSPUR, CA, 94939, US

(21) Appl. No.: 11/307,139

(22) Filed: Jan. 25, 2006

Related U.S. Application Data
(60) Provisional application No. 60/647,369, filed on Jan. 25, 2005.

(51) Int. Cl. A61M 11/00

(52) U.S. Cl. 128/200.14

(57) ABSTRACT

A multi-functional, multi-mode diver communication device provides surface communication and continuous GPS tracking between divers and a dive boat. The disclosed invention facilitates the retrieval of a dive boat that has accidentally left divers behind. Divers in distress are provided with means to activate Personal Locator Beacons and Emergency Position Indicating Radio Beacons and to communicate with potential rescuers on a myriad of radio frequencies.

### CROSS-REFERENCE TO RELATED APPLICATIONS

This application claims the benefit of U.S. Provisional Application No. 60/647,369 filed on Jan. 25, 2005.

### BACKGROUND OF THE INVENTION

(1) Field of the Invention

The present invention relates generally to the field of scuba diving, and in particular to methods and handheld apparatuses used for water surface communication and location detection between scuba divers, dive boat operators, rescue personnel and the general boating community.

(2) Description of the Related Art

While on the surface of the water, communication between divers and knowing the relative location of divers and the dive boat is essential to diver safety. In emergency situations where the dive boat is not available to retrieve the divers or has left the dive area, it is essential to diver safety that short, mid, and long range distress communication capability is available to the divers. In most instances a wayward dive boat may be recalled via surface communication to return to the dive area and retrieve the abandoned divers. Divers who are “left behind” realize their problem while on the surface of the water and need to communicate with boats or others who are at or above the surface of the water.

The related art includes underwater diver to diver and surface to underwater diver communication systems for determining the range and bearing of divers underwater and for signaling divers via acoustics, horns, submerged devices, hydra-phones, transducers and other devices. U.S. Pat. No. 6,791,490 by King discloses a flag/float assembly to support a GPS antenna. U.S. Pat. No. 6,762,678 by Arens discloses a communication device for underwater use wherein the distance between users of the device is measured with a data processor that analyzes location bacons, water salinity, and/or sound waves. While these systems provide communication with and between divers who are underwater, the related art does not provide means for two way monitoring of the position of a dive boat and divers on the water surface or means for surface location or short, mid, and long range two way communication in one simple, compact handheld device.

Thus, there is a need in the art for a compact water surface communication system which allows divers, dive masters and dive boat operators and other device holders to communicate with one another and to communicate with the marine community at large. The present invention facilitates communication with rescue personnel and with typical fisherman and recreational boaters who frequent consumer radio bands such as the VHF Marine Band, Family Service Radio Band and other frequencies. There is a need in the art for means to allow divers and others on the water the ability to quickly communicate their location and avoid the classic problem of being unable to summon a dive boat operator who has prematurely left the dive location.

### BRIEF SUMMARY OF THE INVENTION

The present invention is focused on surface communication and navigation and overcomes shortfalls in the related art by providing simple and efficient means to prevent the occurrence or rectify the occurrence of dive boats inadvertently leaving behind divers. The present invention provides means to issue short, mid, and long range distress communications to rescue personnel and recreational boaters who typically use consumer radio bands. The related art's use of devices for underwater communication and navigation has lead to the proliferation of expensive and delicate devices that fail to address the common safety hazard and well publicized problem of a “diver left behind.”

The present invention overcomes shortfalls in the prior art by providing a relatively inexpensive, robust, and multifaceted surface communication and location notification device. In an emergency situation, an abandoned or distressed diver needs to summon assistance from people who are at or above the surface of the water. Coast Guard rescue personnel are typically dispatched from locations at or above the surface of the water. In a crises situation, means to communicate underwater are of little value.

The present invention provides a novel communication system and method allowing device holders to transmit and receive geographical location information to any similar device and to verbally communicate in a selectable multifunctional transmit and receive audio voice communication mode with any standard VHF Marine Band channel, Family Radio Service (FRS) bands, General Mobile Radio Service (GMRS) channels and other frequencies. The present invention integrates the automatic location and geographical tracking of GPS and selectable multi-mode voice communications of VHF Marine Bands, FRS, GMRS and other frequency bands in a hand held waterproof housing that may be held, placed in a pocket, or attached to a diver's buoyancy control device (BCD).

### BRIEF DESCRIPTION OF THE SEVERAL VIEWS OF THE DRAWINGS

FIG. 1 is a top view the device and illustrates the send and receive tracking function by any similar device and position reporting of other divers.

FIG. 2 is a top view the device and illustrates the send and receive position reporting activation with distance and heading to the dive boat.

FIG. 3 is a top view the device and illustrates the display and function of the VHF marine band channel 16 and FRS channel 6.31/GMRS mode.

FIG. 4 is a top view the device and illustrates the display and function of VHF marine band channel 16 and DSC distress activation and GPS coordinate location for diver in distress location.

FIG. 5 is a top view of the two sides of the waterproof housing.

### DETAILED DESCRIPTION

The following definitions are well known in the art and are used herein.

### DEFINITION LIST 1

| Term | Definition |
|---|---|
| EPIRB | Emergency Position Indicating Radio Beacon |
| DSC | Digital Selective Calling |
| GPS | Global Positioning System |
| PLB | Personal Locator Beacon |
| BCD | Buoyancy Control Device |
| GMRS | General Mobile Radio Service |
| FRS | Family Radio Service |
| VHF Marine | VHF Marine Band Radio Channels and |
| Band Radio | Frequencies |

The following detailed description is directed to certain specific embodiments of the invention. However, the invention can be embodied in a multitude of different ways as defined and covered by the claims. In this description, reference is made to the drawings wherein like parts are designated with like numerals throughout.

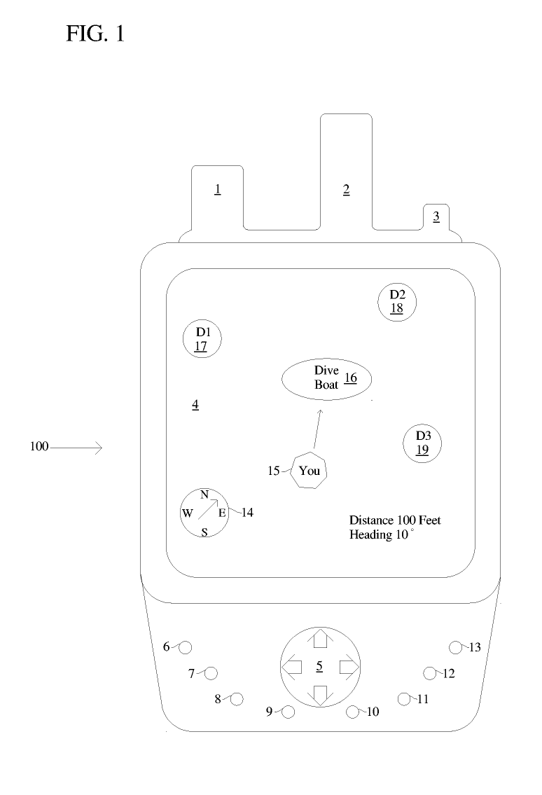

The device, shown on FIG. 1 as generally 100, has multi-band capability to select voice communication with any VHF Marine Band radio and includes FRS/GMRS modes for providing two-way tracking and audio capability. The device may transmit over emergency frequencies including DSC distress calls containing the device GPS position. The device may also transmit and receive over a myriad of frequencies.

The device includes a transmitter and receiver for co-location verification, a transmitter and receiver for two-way multimode audio communications, a transmitter and receiver for standard VHF Marin Band (including DSC), a Global Positions System (GPS-receive/transmit) capability to send and receive GPS positions from other devices for longitude and latitude location of divers and dive boats.

Copies of the disclosed hand held device may mutually interrogate each other to update each device location. The device has the capability to transmit the device location to any other device and to transmit audio and visual distress signals to other devices.

The device display 4 on FIG. 1 may show the device user identification 15, the direction and distance of the sender and receiver, the GPS coordinates of both the dive boat 16, and the divers 17, 18 and 19 on the water surface.

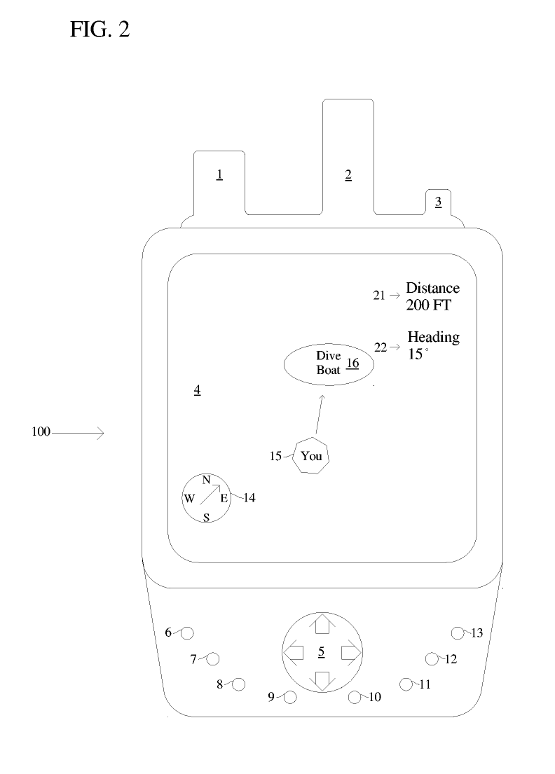

FIG. 2 shows generally the device 100 in a send and receive position reporting mode. The device holder 15 is given distance 21 and heading information 22 to the dive boat 16 and an electronic compass 14 is displayed.

The device includes a GPS antenna 1, a VHF/FRS/GMRS antenna 2, and a DCS antenna 3. A round push wheel 5 allows for selection of navigation and menu functions. Function/mode selection buttons 6,7,8,9,10,11,12, and 13 are on the face of the device.

The round push wheel 5 in combination with the function/mode selection buttons 6-13 allow for communication on a myriad of radio frequencies. A device holder in distress may communicate on consumer frequencies and reach recreational boaters or fishermen who do not typically monitor the designated emergency frequencies. A diver in distress may listen for short, mid, and long range communications between people at sea and interrupt their conversation and request help. By use of the GPS functionality, the diver in distress may communicate his precise GPS coordinates to facilitate a speedy rescue.

The round push wheel 5 in combination with the function/mode selection buttons 6-13 allow for the setting of a “safety distance” between a diver and the dive boat or between divers. The devices continuously interrogate each other and alarm if the safety distance is exceeded. Use of the push wheel 5 in combination with the function/mode selection buttons 6-13 allow for the setting of various alarms to trigger in the event the safety distance is exceeded. The device may also be set to issue DSC, PLB, EPIRB, or other signals in the event the safety distance is exceeded.

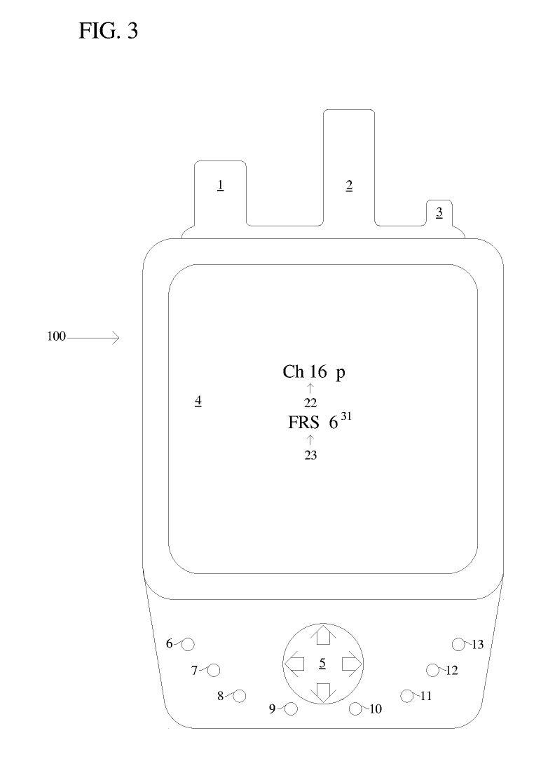

FIG. 3 illustrates the display and function of the VHF marine band channel 16 (at no. 22) and FRS channel 6.31 (at number 23) in GMRS mode.

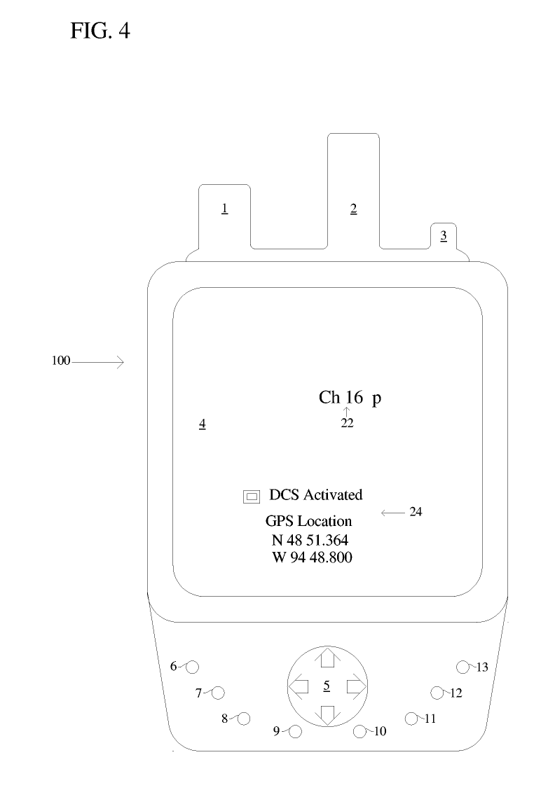

FIG. 4 illustrates the display and function of VHF marine band channel 16 (at no. 22) and DSC distress activation and GPS coordinate location 24 for diver in distress location.

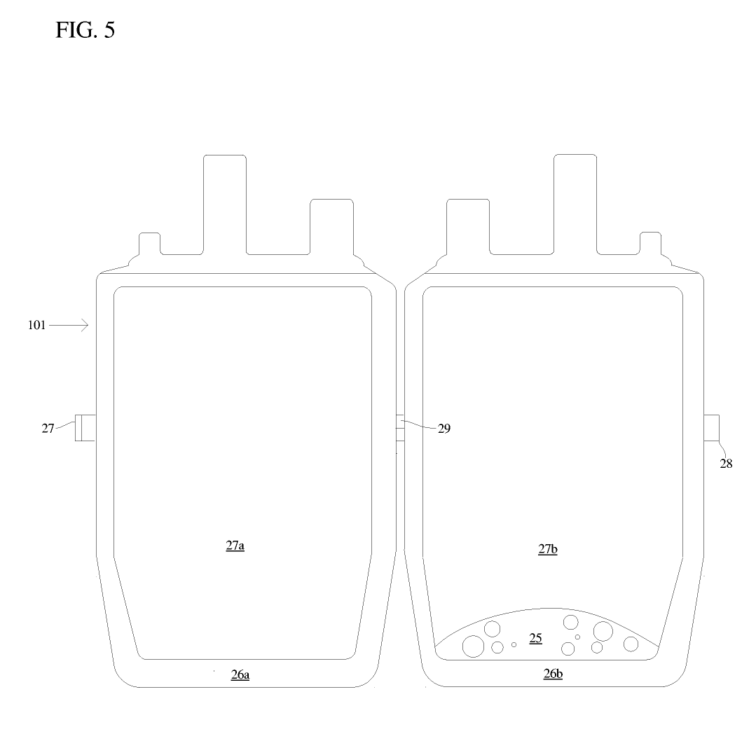

FIG. 5 illustrates a waterproof housing 101 with two sides 27a and 27b that are connected by a hinge 29 and secured by seals 26a and 26b. A latch 27 fits with latch receptacle 28 to secure 27a and 27b. Side 26b may contain weights 25 for neutral buoyancy. The waterproof housing 101 contains the disclosed device and may be carried by hand, stored in a pocket or attached to the body or gear of a diver.

What is claimed is:

1. A method for determining the geographic relationship between one or more divers and a dive boat comprising:
a. initiating and transmitting active and passive selectable signal frequencies, containing device GPS position information, from at least one diver and a dive boat;
b. receiving active or passive alert signals from at least one diver and dive boat; and
c. displaying geographic relationship data from at least one diver and dive boat.

2. The method of claim 1 with initiation of two way communication from one or more divers or a dive boat.

3. The method of claim 2 with communication signals occurring on VHF marine band, Family Radio Service, General Mobile Radio Service, Emergency Position Indicating Radio beacon, Digital Selective Calling or Personal Location Beacon channels.

4. The method of claim 2 with communication signals containing means for distress beacon activation.

5. The method of claim 2 with communication to or from third parties.

6. A method to summon dive boats that have left behind divers comprising:
a. setting a “safety distance” of allowed distance between a diver and a dive boat;
b. initiating and transmitting active and passive selectable signal frequencies, containing device GPS position information, from at least one diver and a dive boat;
c. receiving active or passive alert signals from at least one diver and dive boat;
d. comparing the safety distance between each diver and the dive boat; and
e. notifying the dive boat and divers when the safety distance is exceeded.

7. The method of claim 6 wherein notification includes the activation of a diver's Personal Locator Beacon or Emergency Position Indicating Radio Beacon.

8. The method of claim 6 wherein notification includes audio or visual alarm in the device held by the dive boat operator.

9. A communication device for use at or above the water surface comprising:
a) means for initiating and transmitting active and passive selectable signal frequencies;
b) means for receiving active or passive alert signals; and
c) means for displaying geographic relationship data received from on or more divers or dive boats.

10. The device of claim 9 with means to initiate two way communication.

11. The device of claim 9 with means to mutually interrogate other devices to update each device location.

12. The device of claim 9 wherein the display assembly displays device or user identification, the direction and distance of other devices.

13. The device of claim 9 wherein the display assembly displays GPS coordinates of the device and other devices.

14. The device of claim 9 contained within a waterproof housing.

15. The device of claim 9 with means to attach to a buoyancy control device.

16. The device of claim 9 with means to transmit or receive communication signals occurring on VHF marine band, Family Radio Service, General Mobile Radio Service, Emergency Position Indicating Radio beacon, Digital Selective Calling, or Personal Location Beacon channels.
